(19) United States
(12) Patent Application Publication
(10) Pub. No.: US 2023/0257355 A1
(43) Pub. Date: Aug. 17, 2023

(54) HYDROXAMIC ACID-CONTAINING COMPOUND, AND PREPARATION METHOD AND USE THEREOF

(71) Applicant: CHINA PHARMACEUTICAL UNIVERSITY, Nanjing (CN)

(72) Inventors: Jinxin WANG, Nanjing (CN); Kan YANG, Nanjing (CN); Yu CHEN, Nanjing (CN); Jinying YU, Nanjing (CN); Jibin DONG, Nanjing (CN); Qinlan GU, Nanjing (CN)

(73) Assignee: CHINA PHARMACEUTICAL UNIVERSITY, Nanjing (CN)

(21) Appl. No.: 18/070,529

(22) Filed: Nov. 29, 2022

Related U.S. Application Data
(63) Continuation-in-part of application No. PCT/CN2021/079482, filed on Mar. 8, 2021.

(30) Foreign Application Priority Data
May 29, 2020 (CN) 202010473354.3

(51) Int. Cl. C07D 235/06; C07D 209/08; C07D 333/56

(52) U.S. Cl. CPC C07D 235/06; C07D 209/08; C07D 333/56

(57) ABSTRACT

A hydroxamic acid-containing compound represented by formula I, and a preparation method, and a use thereof are provided. An inhibitory activity of the hydroxamic acid-containing compound on acid sphingomyelinase (ASM) is evaluated by a biological experiment. The compound is further subjected to in vivo pharmacodynamic investigation, and the results show that the compound exhibits significant anti-depression and anti-atherosclerosis (AS) activities, which provides feasibility for the further development of an ASM inhibitor.

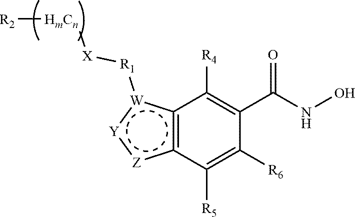

### CROSS REFERENCE TO THE RELATED APPLICATIONS

This application is a continuation-in-part of the national phase entry of International Application No. PCT/CN2021/079482, filed on Mar. 08, 2021, which is based upon and claims priority to Chinese Patent Application No. 202010473354.3, filed on May 29, 2020, the entire contents of which are incorporated herein by reference.

### TECHNICAL FIELD

The present disclosure relates to a hydroxamic acid-containing structural compound, and in particular to a hydroxamic acid-containing compound, and a preparation method and a use thereof.

### BACKGROUND

The hydrolysis of sphingomyelin by acid sphingomyelinase (ASM) is the fastest and most direct pathway for ceramide production in vivo. So far, it has been found that various endogenous and exogenous factors such as tumor necrosis factor-α (TNF-α), interleukin-β (1L-β), and interferon-y (IFN-γ), oxidative stress, ion irradiation, ultraviolet (UV) radiation, thermal shock, trauma, bacterial infection, chemical agents, or the like can activate ASM to cause the massive generation and aggregation of ceramides. A raised ceramide level is involved in the intracellular and extracellular signal transduction and material transfer (FEBS Lett, 2010, 584 (9): 1728-1740).

A large number of studies have shown that the ASM-ceramide pathway is involved in many in vivo processes such as inflammation, apoptosis, and oxidative stress, and is closely correlated with the occurrence and development of various diseases (Progress in Lipid Research, 2016, 61: 51-b2; Apoptosis, 2015, 20: 607-620). It has been found that ASM-involved diseases include atherosclerosis (AS), pulmonary fibrosis, cystic fibrosis (CF), non-alcoholic fatty liver disease (NAFLD), Alzheimer’s disease (AD), multiple sclerosis (MS), depression, or the like (The FASEB Journal, 2008, 22: 3419-3431; Biol. Chem.2015, 396: 707-736).

The restoration of a ceramide level to a normal level by inhibiting ASM can effectively alleviate the symptoms of a related disease. At present, highly-effective specific ASM inhibitors are very lacking, and a small number of direct ASM inhibitors reported in the literature include substrate analogs, diphosphates, and inositol-3,5-diphosphate, which have defects such as poor selectivity, poor drug-likeness, poor stability for phosphatase, and poor membrane permeability and thus cannot be used in the drug development for related diseases (Cell Physiol. Biochem. 2010, 26: 01-08).

Studies have shown that the ASM-ceramide pathway is directly involved in a lesion process of AS. The regulation of metabolic pathways of ceramides is likely to be a potential therapy pathway for AS. The current drugs for treating AS have side effects and poor efficacy. It is of great clinical and scientific research significance to discover a drug with a novel mechanism of action for treating AS, seek for a novel effective therapeutic target, and develop a novel anti-AS drug with ideal clinical efficacy and small side effects.

There are currently no advantageous drugs for treating atypical depression, leading to an increased risk of self-harm and suicide. In addition, clinical antidepressant drugs face serious problems such as slow onset and large side effects. Studies have shown that the inhibition of ASM and the reduction of ceramides play a key role in the development of depression, and an ASM inhibitor can interfere with the ASM-ceramide pathway-mediated signaling. Therefore, the development of a novel antidepressant drug with a novel target and a novel mechanism of action is of great clinical and scientific significance.

ASM is a potential drug target, and there is currently an urgent need to develop a novel direct ASM inhibitor as a candidate drug for treating related diseases.

The present disclosure for the first time discovers and prepares a hydroxamic acid-containing compound, which is a novel compound and is an ASM inhibitor.

### SUMMARY

The present disclosure is intended to provide a hydroxamic acid-containing compound, and a preparation method and a use thereof. The hydroxamic acid-containing compound is a novel ASM inhibitor.

### Technical Solutions

A hydroxamic acid-containing compound with a structure represented by formula I is provided:

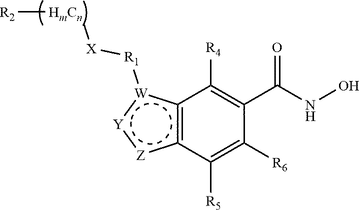

or a pharmaceutically acceptable salt or prodrug of the compound represented by formula I, where
R₁ is selected from the group consisting of C₁-C₁₀ saturated linear alkyl, C₁-C₁₀ unsaturated linear alkyl, C₃-C₁₀ saturated branched alkyl, C₃-C₁₀ unsaturated branched alkyl, and C₃-C₁₀ cycloalkyl, and phenyl;
X is selected from the group consisting of C, N, O, and S;
CₙHₘ is selected from the group consisting of C₁-C₄ saturated linear alkyl, C₁-C₄ unsaturated linear alkyl, C₁~C₄ saturated branched alkyl, and C₁-C₄ unsaturated branched alkyl, where 1 ≤ n ≤ 4 and 2 ≤ m ≤ 8;
R₂ is a monosubstituted or disubstituted phenyl group, in which a substituent is located at an ortho, meta, or para position of a benzene ring and is specifically selected from the group consisting of hydrogen, fluoro, chloro, bromo, cyano, OR₃, CF₃, and SF₅; or R₂ is selected from the group consisting of pyridin-2-yl, pyridin-3-yl, pyridin-4-yl, C₁-C₁₀ saturated linear alkyl, C₁-C₁₀ unsaturated linear alkyl, C₃-C₁₀ saturated branched alkyl, and C₃-C₁₀ unsaturated branched alkyl, where R₃ in OR₃ is selected from the group consisting of H, C₁-C₄ linear or branched alkyl, C₃-C₆ cycloalkyl, and phenyl;
W, Y, and Z are each independently any one selected from the group consisting of C, N, O, and S; and
R₄, R₅, and R₆ are each independently any one selected from the group consisting of H, F, Cl, Br, CF₃, CHF₂, COCF₃, COCH₃, COC₂H₅, CN, C₁-C₆ alkoxy, C₁-C₆ saturated linear alkyl, C₁-C₆ unsaturated linear alkyl, C₃-C₆ saturated branched alkyl, C₃~C₆ unsaturated branched alkyl, and C₃-C₆ cycloalkyl.

In some embodiments of the hydroxamic acid-containing compounds, Ri is selected from the group consisting of phenyl, n-butyl, and n-propyl; R₂ is 3,5-bis(trifluoromethyl)-phenyl, 4-fluorophenyl, 4-cyanophenyl, 4-methoxyphenyl, 2-pyridyl, 2-(5-trifluoromethyl)pyridyl, 4-trifluoromethyl phenyl, 4-methoxy phenyl, 4-trifluoromethoxy phenyl, 4-chlorophenyl, 3-chlorophenyl, 3-bromophenyl, 4-bromophenyl, 2- (5-bromo)pyridyl, 3-fluorophenyl, 2-fluorophenyl, 2-fluoro-4-chlorophenyl, 4-tert-butylphenyl, 3-trifluoromethyl phenyl, 3-thiophenyl, 2-(5-trifluoromethyl)furyl, 2-(5-trifluoromethyl)thiophenyl, 2-(5-chloro)pyridyl, 4-difluoromethoxyphenyl, 2, 4-dichlorophenyl, 3,4,5-trifluoromethyl, heptyl, octyl, nonyl, decyl or the like; X is C or O; W or Z is N; Y is C; and R₄, R₅, and R₆ each are H.

The hydroxamic acid-containing compound has a structure selected from the group consisting of

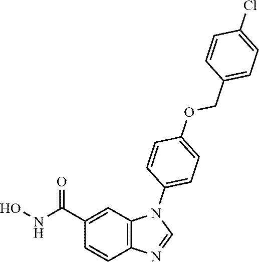 [SMILES: O=C(NO)c1ccc2ncn(-c3ccc(OCc4ccc(Cl)cc4)cc3)c2c1]

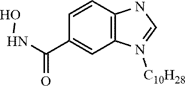

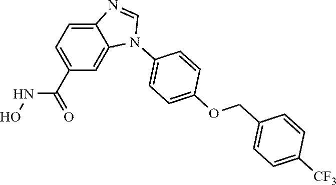 [SMILES: O=C(NO)c1ccc2ncn(-c3ccc(OCc4ccc(C(F)(F)F)cc4)cc3)c2c1]

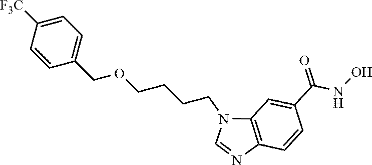 [SMILES: O=C(NO)c1ccc2ncn(CCCCOCc3ccc(C(F)(F)F)cc3)c2c1]

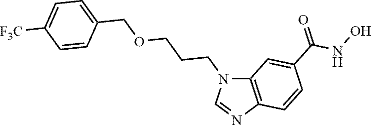 [SMILES: O=C(NO)c1ccc2ncn(CCCOCc3ccc(C(F)(F)F)cc3)c2c1]

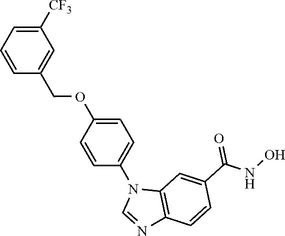 [SMILES: O=C(NO)c1ccc2ncn(-c3ccc(OCc4cccc(C(F)(F)F)c4)cc3)c2c1]

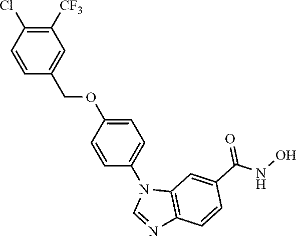 [SMILES: O=C(NO)c1ccc2ncn(-c3ccc(OCc4ccc(Cl)c(C(F)(F)F)c4)cc3)c2c1]

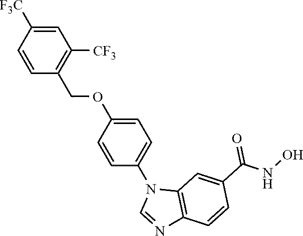 [SMILES: O=C(NO)c1ccc2ncn(-c3ccc(OCc4ccc(C(F)(F)F)cc4C(F)(F)F)cc3)c2c1]

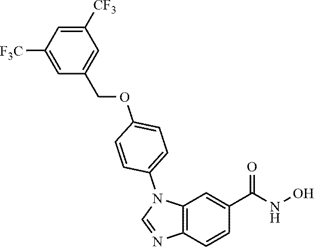 [SMILES: O=C(NO)c1ccc2ncn(-c3ccc(OCc4cc(C(F)(F)F)cc(C(F)(F)F)c4)cc3)c2c1]

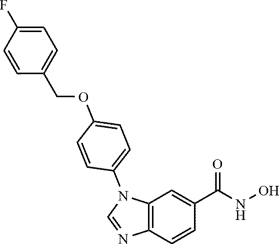 [SMILES: O=C(NO)c1ccc2ncn(-c3ccc(OCc4ccc(F)cc4)cc3)c2c1]

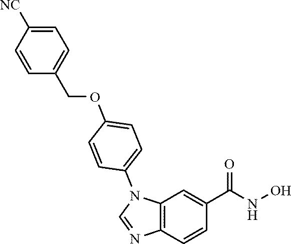 [SMILES: N#Cc1ccc(COc2ccc(-n3cnc4ccc(C(=O)NO)cc43)cc2)cc1]

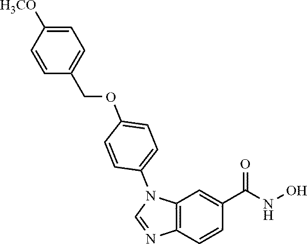 [SMILES: COc1ccc(COc2ccc(-n3cnc4ccc(C(=O)NO)cc43)cc2)cc1]

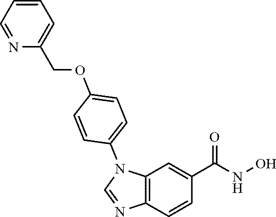 [SMILES: O=C(NO)c1ccc2ncn(-c3ccc(OCc4ccccn4)cc3)c2c1]

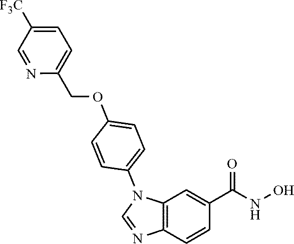 [SMILES: O=C(NO)c1ccc2ncn(-c3ccc(OCc4ccc(C(F)(F)F)cn4)cc3)c2c1]

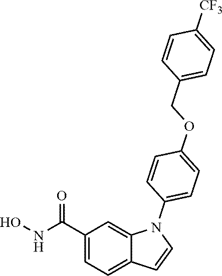 [SMILES: O=C(NO)c1ccc2ccn(-c3ccc(OCc4ccc(C(F)(F)F)cc4)cc3)c2c1]

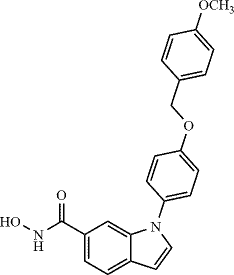 [SMILES: COc1ccc(COc2ccc(-n3ccc4ccc(C(=O)NO)cc43)cc2)cc1]

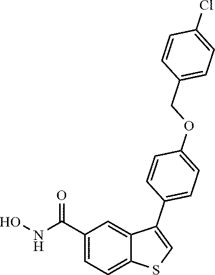 [SMILES: O=C(NO)c1ccc2scc(-c3ccc(OCc4ccc(Cl)cc4)cc3)c2c1]

and

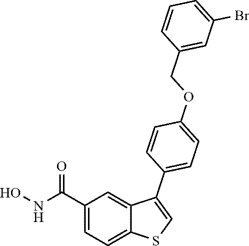 [SMILES: O=C(NO)c1ccc2scc(-c3ccc(OCc4cccc(Br)c4)cc3)c2c1]

Use of the hydroxamic acid-containing compound in the preparation of an ASM inhibitor is provided.

Use of the hydroxamic acid-containing compound in the preparation of a drug for treating a disease selected from the group consisting of AS, diabetes, pulmonary emphysema, pulmonary edema, pulmonary fibrosis, chronic obstructive pulmonary disease (COPD), pulmonary hypertension, CF, non-alcoholic fatty liver disease (NAFLD), AD, MS, stroke, and depression is provided.

### Beneficial Effects

The present disclosure for the first time discovers compounds with a novel skeleton such as compound I1-I14, II-1-II2, and III1-III2. Pharmacodynamic data show that the above compounds are highly potent ASM inhibitors. The compound I-1 is selected for further pharmacodynamic investigation, and the results show that the compound I-1 has prominent anti-depression and anti-AS activities, and thus has promising clinical development potential and application prospects.

### BRIEF DESCRIPTION OF THE DRAWINGS

FIGS. 1A-1D show open field test (OFT) results of compound I-1, where FIG. 1A to FIG. 1D show a total time in center, a times passing the grid, a frequency of upright, and a frequency of grooming, respectively; * indicates p < 0.01 vs Control group; and # indicates p < 0.01 vs Model group.

FIG. 2 shows sucrose preference test (SPT) results of compound I-1.

FIG. 3 shows the reduction of a plaque area in AS mice by compound I-1.

FIGS. 4A-4D show stereograms of aortic arch plaques (AAPs), where FIG. 4A is for a control group 1, FIG. 4B is for a control group 2, FIG. 4C is for a low-dose group, and FIG. 4D is for a high-dose group.

FIG. 5 shows statistical analysis results of volumes of oil red O (ORO)-stained aortic plaques.

FIGS. 6A-6D show images of ORO-stained aortic plaques, where FIG. 6A is for a control group 1, FIG. 6B is for a control group 2, FIG. 6C is for a low-dose group, and FIG. 6D is for a high-dose group.

FIGS. 7A-7D show the influence of compound I-1 on plasma lipid contents, where FIG. 7A is for total triglyceride (TG), FIG. 7B is for total cholesterol, FIG. 7C is for high-density lipoprotein (HDL), and FIG. 7D is for low-density lipoprotein (LDL).

### DETAILED DESCRIPTION OF THE EMBODIMENTS

### Example 1 Synthesis of Compound I

1. Preparation of ethyl 3-((4-hydroxyphenyl)amino)-4-nitrobenzoate (3a)

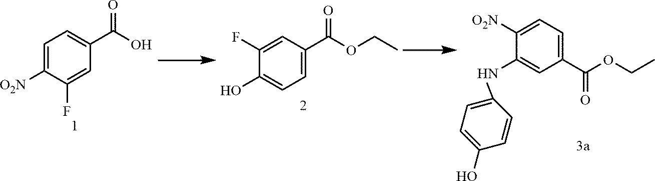

1 g (5.4 mmol) of 3-fluoro-4-nitrobenzoic acid was dissolved in 10 mL of ethanol, thionyl chloride (1.2 mL, 16.2 mmol) was slowly added dropwise, and the resulting mixture was heated at 80° C. for reflux condensation to allow a reaction to occur for 3 h, then cooled to room temperature, and concentrated by rotary evaporation; 40 mL of saturated sodium bicarbonate was added to a reaction flask, followed by extraction with 100 mL of ethyl acetate three times; the combined organic phase was dried over anhydrous sodium sulfate, and subjected to suction filtration, and the resulting filtrate was spin-dried to obtain a light yellow crude intermediate 1; the intermediate 1 was dissolved in 15 mL of N,N-dimethylformamide (DMF), 708 mg (6.5 mmol) of p-hydroxyaniline and 1,636 mg of triethylamine (TEA) were added, and the resulting mixture was heated at 110° C. for reflux condensation to allow a reaction to occur for 6 h and then cooled to room temperature; 40 mL of a 10% dilute HCl solution was added to the resulting reaction solution, followed by extraction with 100 mL of ethyl acetate three times; and the combined organic phase was dried over anhydrous sodium sulfate, and purified through column chromatography (petroleum ether : ethyl acetate = 16:1) to obtain 1.2 g of a red solid (3a), with a yield of 73.6%.

¹H NMR (400 MHz, DMSO-d6) δ 9.64 (s, 1H), 9.38 (s, 1H), 8.21 (dd, J = 8.9, 3.1 Hz, 1H), 7.49 (d, J = 1.7 Hz, 1H), 7.30-7.06 (m, 2H), 7.00-6.72 (m, 1H), 4.26 (q, J = 7.1 Hz, 1H), 1.24 (t, J = 7.0 Hz, 3H), ESI-MS m/z: 303.1 [M+H]⁺

2. Preparation of ethyl 4-amino-3-((4-hydroxyphenyl)amino)benzoate (4a)

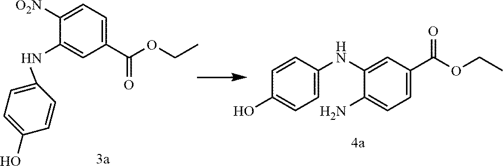

1.2 g (3.97 mmol) of a raw material was dissolved in 15 mL of ethanol, 3 mL of acetic acid and 1,578 mg (23.82 mmol) of zinc powder were added, and the resulting mixture was stirred at room temperature for 12 h; after completion of the reaction as monitored by thin-layer chromatography (TLC), the resulting reaction mixture was subjected to suction filtration; the resulting filter cake was washed until no fluorescence, and the resulting filtrate was spin-dried to obtain a gray-green crude product; and the crude product was recrystallized in a PE/EA system to obtain 1 g of an off-white solid (4a) with a yield of 92.6%.

¹H NMR (400 MHz, DMSO-d6) δ 8.90 (s, 1H), 7.44 (d, J = 1.8 Hz, 1H), 7.35 (dd, J = 6.6, 1.7 Hz, 1H), 6.77-6.61 (m, 6H), 5.55 (s, 2H), 4.17 (q, 2H), 1.23 (t, = 7.1 Hz, 3H), ESI-MS m/z: 273.1 [M+H]⁺

3. Preparation of ethyl 1-(4-hydroxyphenyl)-1H-benzo[d]imidazole-6-carboxylate (5a)

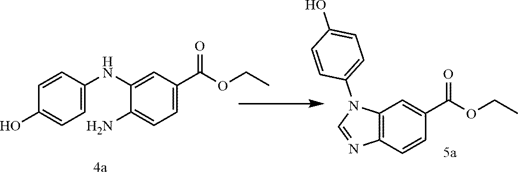

500 mg (1.84 mmol) of a raw material was dissolved in 3.8 g (36 mmol) of dried trimethyl orthoformate (TMOF), and the resulting solution was heated at 110° C. for reflux condensation to allow a reaction to occur for 4 h; after completion of the reaction as monitored by TLC, the resulting reaction mixture was cooled to room temperature, followed by rotary evaporation to remove a part of the solvent; 40 mL of 10% dilute hydrochloric acid was added, and the resulting mixture was shaken for 5 min, extracted with 100 mL of ethyl acetate three times, and washed with a saturated sodium chloride solution twice; and the combined organic phase was dried over anhydrous sodium sulfate, and purified through column chromatography (petroleum ether : ethyl acetate = 4:1) to obtain 378 mg of a light-brown solid (5a) with a yield of 65.1%. ¹H NMR (300 MHz, DMSO-d₆) δ 8.76 (s, 1H), 8.12 (dd, J = 7.5, 1.6 Hz, 1H), 8.04-7.99 (m, 2H), 7.70 (d, 1H), 7.61 - 7.56 (m, 2H), 6.88-6.83 (m, 2H), 4.33 (q, 2H), 1.37 (t, 3H), ESI-MS m/z: 283.1 [M+H]⁺

4. Preparation of ethyl 1-(4-((4-chlorobenzyl)oxy)phenyl)-1H-benzo[d]imidazole-6-carboxylate (6a)

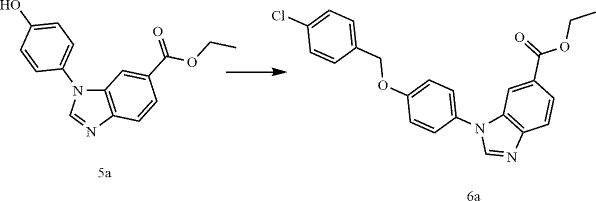

200 mg (0.72 mmol) of a raw material and 176 mg (0.86 mmol) of p-chlorobenzyl bromide were dissolved in 10 mL of acetone, 702 mg (2.16 mmol) of cesium carbonate and a catalytic amount of potassium iodide were added, and the resulting mixture was heated at 65° C. for reflux condensation to allow a reaction to occur for 12 h; and after completion of the reaction as monitored by TLC, the resulting reaction mixture was subjected to suction filtration and purified through column chromatography (petroleum ether : ethyl acetate = 2:1) to obtain 168 mg of a yellow solid with a yield of 59.6%. ¹H NMR (300 MHz, DMSO-d₆) δ 8.73 (s, 1H), 8.07 (s, 1H), 7.93 (q, J = 8.6 Hz, 2H), 7.54 (t, J = 7.5 Hz, 6H), 7.32 (d, J = 8.7 Hz, 2H), 5.27 (s, 2H), 4.36 (q,J = 7.1 Hz, 2H), 1.35 (t, J = 7.0 Hz, 3H). ESI-MS m/z: 407.1 [M+H]⁺

5. Preparation of 1-(4-((4-chlorobenzyl)oxy)phenyl)-N-hydroxy-1H-benzo[d]imidazole-6-carboxamide (I-1)

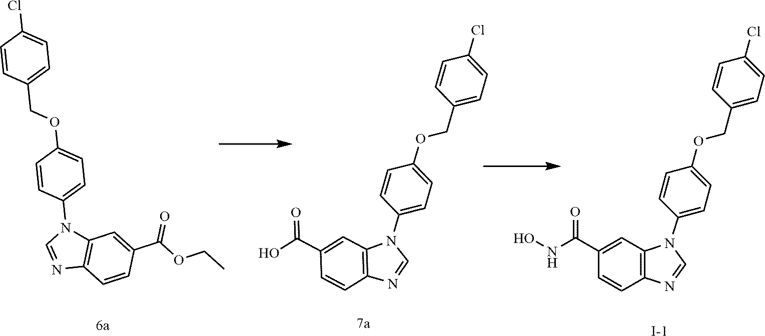

Step 1: 168 mg (0.41 mmol) of a raw material was dissolved in 4 mL of methanol, 4 mL of H₂O and 82 mg (2.05 mmol) of NaOH were added, and the resulting mixture was heated at 80° C. for reflux condensation to allow a reaction to occur for 2 h; after completion of the reaction as monitored by TLC, 20 mL of 10% HCl was added to precipitate a white solid product 7a; the white solid product 7a was dried and dissolved in 10 mL of dried anhydrous dichloromethane (DCM), 0.1 mL of thionyl chloride was added with a pipette to the reaction flask, and the resulting mixture was heated at 45° C. under nitrogen protection for reflux condensation to allow a reaction to occur for 3 h; and after completion of the reaction as monitored by TLC, the solvent was removed by rotary evaporation. Step 2: Another reaction flask was prepared, 144 mg (2.05 mmol) of hydroxylamine hydrochloride, 82 mg (2.05 mmol) of sodium hydroxide, 8 mL of tetrahydrofuran (THF), and 0.5 mL of H₂O were added, and the resulting mixture was stirred at room temperature. Step 3: The product obtained in step 2 was dissolved in 5 mL of anhydrous THF, the resulting solution was slowly added dropwise with a constant-pressure dropping funnel to the reaction flask, and then the resulting mixture was stirred at room temperature for 3 h to allow a reaction to occur; and after completion of the reaction as monitored by TLC, the PH of the resulting reaction solution was adjusted with 10% dilute HCl to neutral to precipitate a large amount of a white solid, and the resulting mixture was refrigerated for a period of time and then subjected to suction filtration to obtain 98 mg of a white solid product with a yield of 61%. ¹H NMR (500 MHz, Chloroform-d) δ 8.79 (d, J = 4.9 Hz, 1H), 8.18 (d, J = 1.5 Hz, 1H), 8.05 (s, 1H), 7.82 (dd, J = 7.5, 1.5 Hz, 1H), 7.78 (d, J = 7.4 Hz, 1H), 7.62-7.57 (m, 2H), 7.42 (d, J = 0.8 Hz, 4H), 7.03-6.97 (m, 2H), 5.05 (s, 2H). ESI-MS m/z: 392.1 [M-H]⁻

### Other Compounds Were Synthesized With Reference to Example 1

I-2

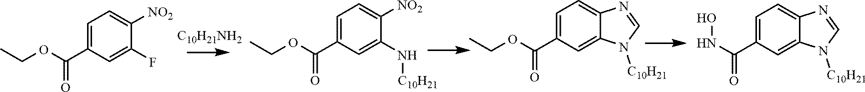

According to the method for constructing benzopyrazole skeleton of I-1, a decane chain was first introduced by a substitution reaction, then a cyclization reaction was performed, and then 460 mg of a white solid product (I-2) was prepared with reference to the synthesis method of I-1, with a yield of 71%. ¹H NMR ((400 MHz, DMSO-d₆δ 11.41 (s, 1H), 9.27 (s, 1H), 8.48 (s, 1H), 8.26 (s, 1H), 7.8 (m, 2H), 4.24 (t, J=7.1 Hz, 2H), 1.40-1.22 (m, 16H), 0.93-0.84 (m, 3H), ESI-MS m/z: 318.2 [M+H]⁺

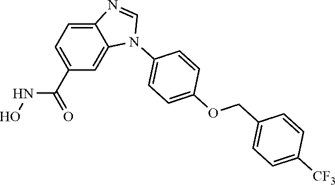 [SMILES: O=C(NO)c1ccc2ncn(-c3ccc(OCc4ccc(C(F)(F)F)cc4)cc3)c2c1]

779 mg of a white solid (I-3) was prepared with reference to the synthesis method of I-1, with a yield of 64%. ¹H NMR (400 MHz, DMSO-d₆) δ 11.45 (s, 1H), 9.33 (s, 1H), 8.22-6.28 (m, 12H), 5.41 (s, 2H), ESI-MS m/z: 428.2 [M+H]⁺

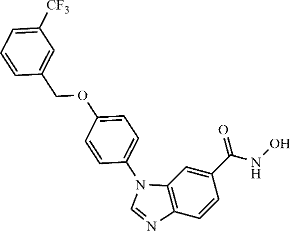 [SMILES: O=C(NO)c1ccc2ncn(-c3ccc(OCc4cccc(C(F)(F)F)c4)cc3)c2c1]

228 mg of a white solid product (I-6) was prepared with reference to the synthesis method of I-1, with a yield of 59%. ¹H NMR (400 MHz, DMSO-d₆) δ 11.40 (s, 1H), 9.34 (s, 1H), 8.12-7.01 (m, 12H), 5.36 (s, 2H), ESI-MS m/z: 428.1 [M+H]⁺

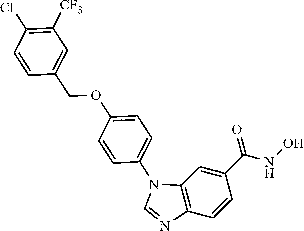 [SMILES: O=C(NO)c1ccc2ncn(-c3ccc(OCc4ccc(Cl)c(C(F)(F)F)c4)cc3)c2c1]

150 mg of an off-white solid product (I-7) was prepared with reference to the synthesis method of I-1, with a yield of 56%. ¹H NMR (400 MHz, DMSO-d₆) δ 11.29 (s, 1H), 9.03 (s, 1H), 8.79-6.96 (m, 11H), 5.48 (s, 2H), ESI-MS m/z: 462.0 [M+H]⁺

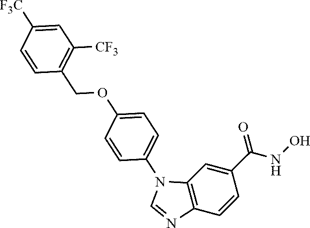 [SMILES: O=C(NO)c1ccc2ncn(-c3ccc(OCc4ccc(C(F)(F)F)cc4C(F)(F)F)cc3)c2c1]

144 mg of an off-white solid product was prepared with reference to the synthesis method of I-1, with a yield of 57%. ¹H NMR (400 MHz, DMSO-d₆) δ 11.36 (s, 1H), 9.15(s, 1H), 8.82-7.06 (m, 11H), 5.44 (s, 2H), ESI-MS m/z: 494.0 [M-H]⁻.

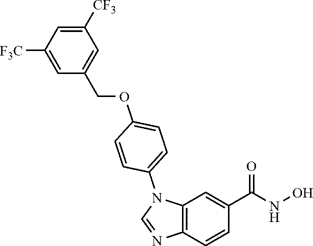 [SMILES: O=C(NO)c1ccc2ncn(-c3ccc(OCc4cc(C(F)(F)F)cc(C(F)(F)F)c4)cc3)c2c1]

118 mg of a gray-blue powdery solid product was prepared with reference to the synthesis method of I-1, with a yield of 60%. M.P. 238-240° C. ¹H NMR (300 MHz, DMSO-d6) δ 11.48 (s, 1H), δ 9.42 (s, 1H), 8.26-7.39 (m, 11H), 5.47 (s, 2H) ppm; ¹³ C NMR (101 MHz, DMSO) δ 163.91, 158.87, 144.69, 140.79, 132.59, 131.08, 130.75, 130.43, 130.16, 128.88, 128.05, 127.01, 125.14, 124.17, 122.43, 117.59, 116.63, 111.38, 68.58, 40.61, 40.40, 40.19, 39.98, 39.77, 39.57, 39.36 ppm; HRMS (ESI+): m/z [M+H]+ calcd for C23H15F6N3O3, 496.1018; found 496.1089.

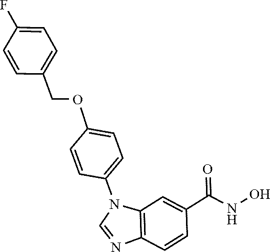 [SMILES: O=C(NO)c1ccc2ncn(-c3ccc(OCc4ccc(F)cc4)cc3)c2c1]

80 mg of a light-blue powdery solid product was prepared with reference to the synthesis method of I-1, with a yield of 54%. M.P. 235-237° C. ¹H NMR (400 MHz, DMSO-d6) δ 9.62 (s, 1H), 8.03-7.26 (m,12H), 5.24 (s, 2H) ppm; 13C NMR (101 MHz, DMSO) δ 163.64, 163.55, 161.13, 159.45, 144.31, 133.35, 133.31, 132.30, 130.63, 130.59, 130.55, 127.24, 127.14, 124.85, 116.90, 116.55, 115.96, 115.75, 111.68, 69.44, 40.63, 40.58, 40.42, 40.37, 40.21, 40.16, 39.95, 39.74, 39.53, 39.33 ppm; HRMS (ESI+): m/z [M+H]⁺ calcd for C21H16FN3O3, 378.1276, found 378.1251.

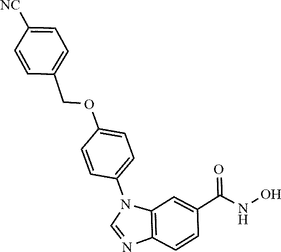

10 mg of a light-yellow powdery solid product was prepared with reference to the synthesis method of I-1, with a yield of 40%. M.P. 220-222° C. ¹H NMR (400 MHz, DMSO-d6) δ 10.23 (s, 1H), 9.10 (s, 1H), 7.98-7.31 (m,12H), 5.37 (s, 2H) ppm; HRMS (ESI+): m/z [M+H]⁺ calcd for C22H16N4O3, 385.1204; found 385.1222.

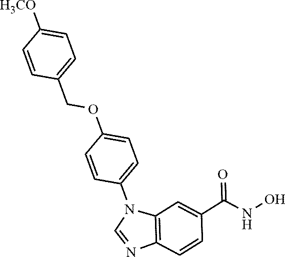 [SMILES: COc1ccc(COc2ccc(-n3cnc4ccc(C(=O)NO)cc43)cc2)cc1]

80 mg of a gray-blue solid product was prepared with reference to the synthesis method of I-1, with a yield of 62%. M.P. 240-242° C. ¹H NMR (400 MHz, DMSO-d6) δ 10.43 (s, 1H), 9.53(s, 1H), 8.15-7.05 (m, 12H), 5.25 (s, 2H), 3.86 (s, 3H) ppm; ¹³ C NMR (101 MHz, DMSO) δ 163.71, 159.60, 159.58, 144.30, 132.35, 130.53, 130.10, 128.94, 127.14, 127.05, 124.76, 116.99, 116.56, 114.39, 111.65, 69.97, 55.62, 40.61, 40.40, 40.19, 39.98, 39.77, 39.57, 39.36 ppm; HRMS (ESI+): m/z [M+H]⁺ calcd for C22H19N3O4, 390.1476; found 390.1453.

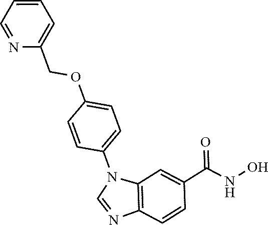 [SMILES: O=C(NO)c1ccc2ncn(-c3ccc(OCc4ccccn4)cc3)c2c1]

70 mg of a light-green powdery solid product was prepared with reference to the synthesis method of I-1, with a yield of 56%. ¹H NMR (300 MHz, DMSO-d6) δ 10.31 (s, 1H), 9.16 (s, 1H), 8.71 (d, J = 4.8 Hz, 1H), 8.11-7.29 (m, 11H), 5.41 (s, 2H) ppm; HRMS (ESI+): m/z [M+H]⁺ calcd for C22H19N3O4, 390.1476; found 390.1453.

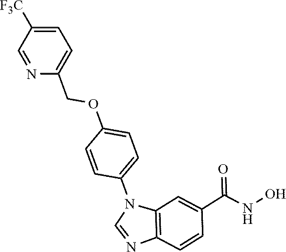 [SMILES: O=C(NO)c1ccc2ncn(-c3ccc(OCc4ccc(C(F)(F)F)cn4)cc3)c2c1]

A light-green powdery solid product was prepared with reference to the synthesis method of I-1, with a yield of 52%. M.P. 230-232° C. ¹H NMR (300 MHz, DMSO-d6) δ 9.65 (d, J = 3.5 Hz, 1H), 8.54 (d, J = 1.9 Hz, 1H), 8.22-8.11 (m, 2H), 7.96 (dd, J = 6.9, 1.9 Hz, 1H), 7.89 (dd, J = 7.9, 2.2 Hz, 1H), 7.82 (dd, J = 7.9, 0.5 Hz, 1H), 7.61-7.52 (m, 3H), 7.10-7.01 (m, 2H), 5.18 (s, 2H) ppm.

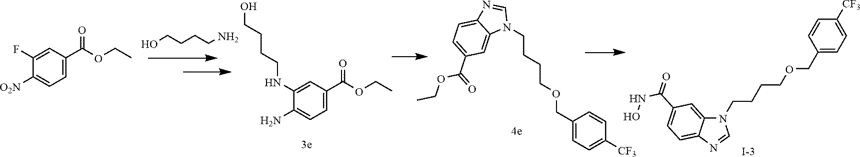

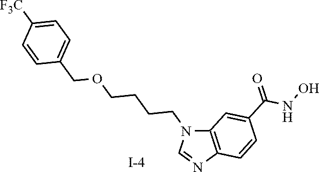

According to the method for synthesizing benzopyrazole skeleton of I series, a substitution reaction was performed with 4-amino-1-butanol, the resulting product was then reduced to 3e, the 3e was subjected to a cyclization reaction and then to a substitution reaction with 4-trifluoromethylbenzyl bromide to obtain 4e, and 112 mg of a red solid product (I-4) was prepared with reference to the synthesis method of I-1, with a yield of 47% ¹H NMR( 400 MHz, DMSO-d₆) δ 11.27 (s, 1H), 9.17 (s, 1H), 8.26 (s, 1H), 8.02 (s, 1H), 7.77 (d, J = 1.3 Hz, 2H), 7.64-7.58 (m, 2H), 7.31 (m, 2H), 4.47 (t, J = 1.0 Hz, 2H), 4.29 (t, 2H), 3.48 (t, J = 7.1 Hz, 2H), 1.96 (m, 2H), 1.79 (m, 2H), ESI-MS m/z: 408.2 [M+H]⁺.

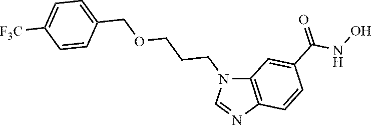 [SMILES: O=C(NO)c1ccc2ncn(CCCOCc3ccc(C(F)(F)F)cc3)c2c1]

86 mg of a red solid product (I-5) was prepared with reference to the synthesis method of I-4, with a yield of 41%. ¹H NMR (400 MHz, DMSO-d₆) δ 11.29 (s, 1H), 9.05 (s, 1H), 8.37 (s, 1H), 8.09 (s, 1H), 7.70 (m, 2H), 7.54 (d, J = 7.9 Hz, 2H), 7.45 (s, 1H), 7.33 (s, 1H), 7.20 (s, 1H), 4.55 (m, 6H), 1.23 (s, 2H), ESI-MS m/z: 394.1 [M+H]⁺.

### Example 2 Synthesis of Compound II

1. Preparation of ethyl 1H-indole-6-carboxylate (8a)

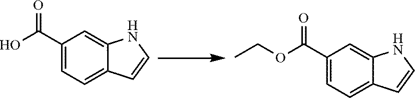

1 g (6.2 mmol) of 1H-indole-6-carboxylic acid was dissolved in 15 mL of absolute ethanol, 0.2 mL of concentrated sulfuric acid was slowly added dropwise, and the resulting mixture was refluxed for 20 h; after completion of the reaction as monitored by TLC, the resulting reaction mixture was concentrated by rotary evaporation, 40 mL of a saturated sodium bicarbonate solution was added to the reaction flask, followed by extraction with 100 mL of ethyl acetate three times; and the combined organic phase was dried over anhydrous sodium sulfate, and subjected to suction filtration, and the filtrate was spin-dried to obtain a light-yellow crude intermediate 8a, with a yield of 81%. ¹H NMR (300 MHz, DMSO-d6) δ 11.93 (s, 1H), 8.07 (d, J = 30 Hz, 1H), 7.99 (m, 1H), 7.47 (m, 1H), 7.19 (m, 2H), 4.27 (q, J = 7.1 Hz, 2H), 1.33 (t, J = 7.1 Hz, 3H) ppm.

2. Preparation of ethyl l-(4-methoxyphenyl)-indole-6-carboxylate (9a)

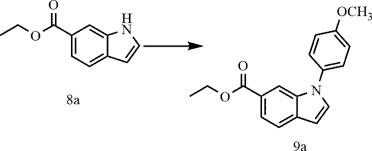

500 mg (2.6 mmol) of the intermediate 8a was dissolved in 10 mL of N-methylpyrrolidone (NMP), K₂CO₃ (719 mg, 5.2 mmol), CuBr (19 mg, 0.52 mmol), and 4-bromoanisole (972 mg, 0.52 mmol) were added successively, and a reaction was allowed to occur at 170° C. for 20 h; after completion of the reaction as monitored by TLC, 40 mL of a saturated sodium chloride solution was added to the reaction flask, followed by extraction with 100 mL of ethyl acetate three times; and the combined organic phase was dried over anhydrous sodium sulfate, and subjected to suction filtration, and the filtrate was spin-dried and purified through column chromatography to obtain a white crude intermediate 9a, with a yield of 75%. ¹H NMR (300 MHz, DMSO-d6) δ 8.21 (m, 1H), 8.13 (m, 1H), 7.43 (m, 3H), 7.29 (m, 2H), 7.10 (m, 2H), 4.29 (q, J = 7.0 Hz, 2H), 3.83 (s, 3H), 1.35 (t, J = 7.0 Hz, 3H) ppm.

3. Preparation of ethyl 1-(4-hydroxyphenyl)-indole-6-carboxylate (10a)

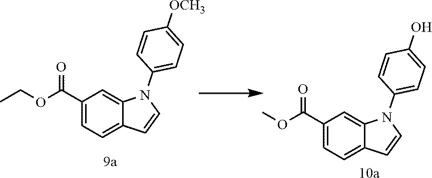

500 mg (1.7 mmol) of the intermediate 9a was dissolved in 10 mL of DCM, BBr₃ (0.6 mL, 12.0 mmol) was added dropwise at 0° C., and a reaction was allowed to occur for 6 h under the protection of N₂, after completion of the reaction as monitored by TLC, 5 mL of absolute methanol was added to the reaction flask for quenching, followed by extraction with 100 mL of DCM three times; and the combined organic phase was dried over anhydrous sodium sulfate, and subjected to suction filtration, and the filtrate was spin-dried and purified through column chromatography to obtain a light-yellow crude intermediate 10a, with a yield of 64%. ¹H NMR (300 MHz, DMSO-d6) δ 9.90 (s, 1H), 8.19 (s, 1H), 8.10 (m, 1H), 7.41 (m, 3H), 7.27 (m, 2H), 6.96 (m, 2H), 3.83 (s, 3H) ppm.

4. Preparation of 1-(4-((4-trifluoromethylbenzyl)oxy)phenyl)-N-hydroxy-1H-indole-6-carboxamide (II-1)

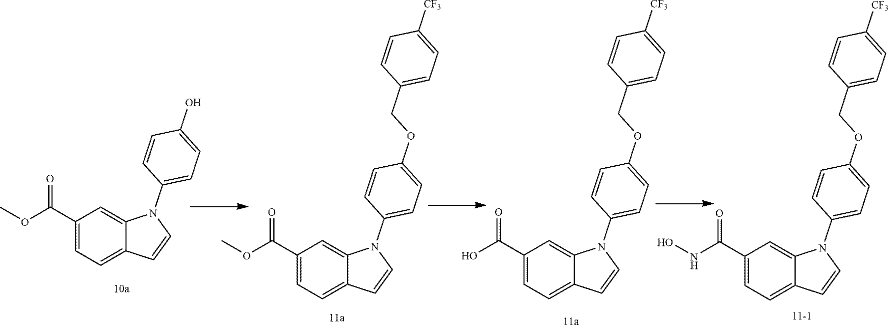

A brown solid product was prepared with reference to the synthesis method of I-1, with a yield of 54%. ¹H NMR (300 MHz, DMSO-d6) 810.75 (s, 1H), 8.95 (s, 1H), 8.20 (d, J = 7.4 Hz, 1H), 8.07 (s, 1H), 7.80 (d, J = 7.4 Hz, 2H), 7.72 (d, J = 7.3 Hz, 2H), 7.54 (d, J = 7.6 Hz, 2H), 7.41 (m, 11H), 7.25 (m, 4H), 5.33 (s, 2H) ppm.

5. Preparation of 1-(4-((4-methoxybenzyl)oxy)phenyl)-N-hydroxy-1H-indole-6-carboxamide (II-2)

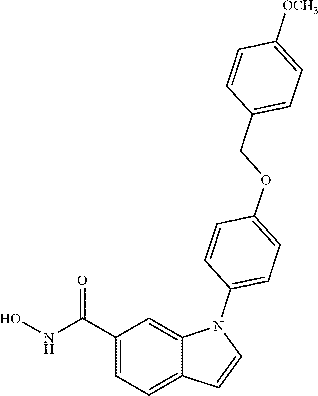 [SMILES: COc1ccc(COc2ccc(-n3ccc4ccc(C(=O)NO)cc43)cc2)cc1]

A brown solid product was prepared with reference to the synthesis method of I-1, with a yield of 19%. ¹H NMR (300 MHz, DMSO-d6) δ10.73 (s, 1H), 8.92 (s, 1H), 8.20 (s, 1H), 8.08 (m, 1H), 7.50 (d, J = 6.7 Hz, 2H), 7.41 (m, 3H), 7.23 (m, 4H), 6.97 (d, J = 6.4 Hz, 2H), 5.10 (s, 2H), 3.76 (s, 3H) ppm.

### Example 3 Synthesis of Compound III

1. Preparation of benzo[b]thiophene-5-carbonitrile

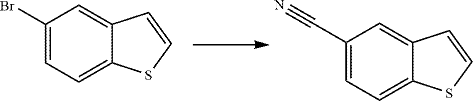 [SMILES: Brc1ccc2sccc2c1 ; N#Cc1ccc2sccc2c1]

1.0 g (4.7 mmol) of 5-bromobenzo[b]thiophene was dissolved in 15 mL of DMF, then a catalytic amount of Pd(PPh₃)₄ and 1.1 g (9.4 mmol) of Zn(CN)₂ were added successively, and a reaction was allowed to occur for 10 h under the protection of N₂; after completion of the reaction as monitored by TLC, 40 mL of a saturated saline solution was added to the reaction flask, followed by extraction with 100 mL of ethyl acetate three times; and the combined organic phase was dried over anhydrous sodium sulfate, followed by rotary evaporation, and purified through column chromatography to obtain a crude intermediate 9a.

2. Preparation of benzo[b]thiophene-5-carboxylic acid

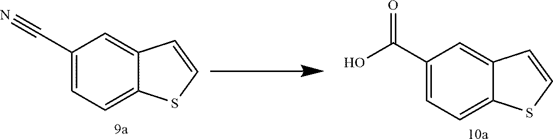

500 mg (3.14 mmol) of the intermediate 9a and 750 mg (3.14 mmol) of LiOH were dissolved in a mixed solvent of methanol and water in a volume ratio of 1:1, and the resulting mixture was heated to allow a reaction to occur for 6 h; after completion of the reaction as monitored by TLC, dilute hydrochloric acid was added dropwise to adjust the PH to 3 to 4, followed by extraction with 100 mL of ethyl acetate three times; and the combined organic phase was dried over anhydrous sodium sulfate, followed by rotary evaporation, and purified through column chromatography to obtain a crude intermediate 10a.

3. Preparation of ethyl benzo[b]thiophene-5-carboxylate

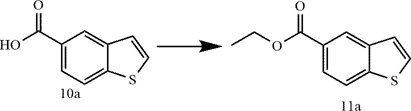

1 g (5.6 mmol) of the intermediate 10a was dissolved in 15 mL of absolute ethanol, 0.2 mL of concentrated sulfuric acid was slowly added dropwise, and the resulting mixture was refluxed for 20 h; after completion of the reaction as monitored by TLC, the resulting reaction mixture was concentrated by rotary evaporation, 40 mL of a saturated sodium bicarbonate solution was added to the reaction flask, followed by extraction with 100 mL of ethyl acetate three times; and the combined organic phase was dried over anhydrous sodium sulfate, and subjected to suction filtration, and the filtrate was spin-dried to obtain a crude intermediate 11a.

4. Preparation of ethyl 3-bromo-benzo[b]thiophene-5-carboxylate

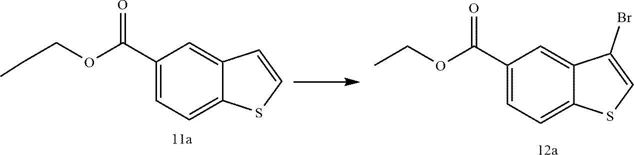

500 mg (2.4 mmol) of the intermediate 11a was dissolved in 12 mL of acetonitrile, 432 mg (4.8 mmol) of N-Bromosuccinimide (NBS) was added, and the resulting mixture was stirred at room temperature for 30 min; after completion of the reaction as monitored by TLC, 40 mL of a saturated sodium bicarbonate solution was added to the reaction flask, followed by extraction with 100 mL of ethyl acetate three times; and the combined organic phase was dried over anhydrous sodium sulfate, and spin-dried by rotary evaporation, to obtain a crude intermediate 12a, which was directly used in the next reaction without purification.

5. Preparation of ethyl 3-(4-hydroxyphenyl)benzo[b]thiophene-5-carboxylate

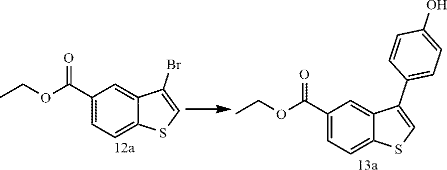

1 g (3.5 mmol) of the intermediate 12a was dissolved in 15 mL of a dioxane solution, 975 mg (7.0 mmol) of 4-hydroxyphenylboronic acid, 975 mg (7.0 mmol) of anhydrous potassium carbonate, and a catalytic amount of Pd(dppf)Cl₂ were added, and a reaction was allowed to occur at 110° C. for 12 h; after completion of the reaction as monitored by TLC, 40 mL of a saturated saline solution was added to the reaction flask, followed by extraction with 100 mL of ethyl acetate three times; and the combined organic phase was dried over anhydrous sodium sulfate, spin-dried by rotary evaporation, and purified through column chromatography to obtain an intermediate 13a as a light-yellow solid, with a yield of 43%. ¹H NMR (300 MHz, DMSO) δ 9.75 (s, 1H), 8.02 (t, J = 4.1 Hz, 2H), 7.58 (t, J = 7.8 Hz, 1H), 7.39 (d, J = 7.8 Hz, 3H), 6.95 (d, J = 8.1 Hz, 2H), 3.87 (s, 3H).

6. Preparation of 4-(4-((4-chlorobenzyl)oxy)phenyl)-N-hydroxybenzo[b]thiophene-2-carboxamide (III-1)

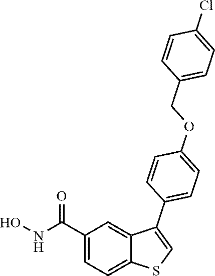 [SMILES: O=C(NO)c1ccc2scc(-c3ccc(OCc4ccc(Cl)cc4)cc3)c2c1]

38 mg of a pink solid was prepared with reference to the synthesis method of I-1, with a yield of 52%. ¹H NMR (300 MHz, DMSO) δ 11.48 (s, 1H), 9.19 (s, 1H), 8.03 - 7.96 (m, 2H), 7.49 (q, J = 8.2, 7.3 Hz, 7H), 7.41 (d, J = 7.2 Hz, 1H), 7.15 (d, J = 7.9 Hz, 2H), 5.36 (s, 2H).

7. Preparation of 4-(4-((4-bromobenzyl)oxy)phenyl)-N-hydroxybenzo[b]thiophene-2-carboxamide (III-2)

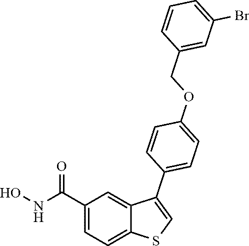 [SMILES: O=C(NO)c1ccc2scc(-c3ccc(OCc4cccc(Br)c4)cc3)c2c1]

40 mg of a pink solid was prepared with reference to the synthesis method of I-1, with a yield of 59%. ¹H NMR (300 MHz, DMSO) δ 11.21 (s, 1H), 9.15 (s, 1H), 8.45 - 7.90 (m, 6H), 7.55 - 7.15 (m, 6H), 5.45 (s, 2H).

### Example 4 Experiment on Inhibition of Compounds on ASM Activity

ASM can hydrolyze sphingomyelin to produce ceramides in cells. For a specified amount of a fluorescently-labeled reaction substrate (Avanti Corporation, USA), different ASM activities catalyze the generation of a product at different amounts. Thus, an ASM activity level can be investigated by detecting a product content. The experiment design of the present disclosure is carried out according to the above principle.

A protein in cultivated cells was extracted, a buffer and a fluorescently-labeled reaction substrate were added, and then the compounds I-1 to I-8 each were separately added at different concentrations. A blank control group was set. After the reaction was completed, fluorescence analysis was performed, and finally an IC₅₀ value of the compound was calculated.

Test results of inhibition of compounds I-1 to I-8 of the present disclosure on the ASM activity

| Compound | IC₅₀ (µM) | Compound | IC₅₀ (µM) |
|---|---|---|---|
| I-1 | 0.47 | I-5 | 2.78 |
| I-2 | 6.58 | I-6 | 1.24 |
| I-3 | 3.68 | I-7 | 0.39 |
| I-4 | 1.34 | I-8 | 1.16 |

A protein was extracted from the cultivated cells, a buffer and a fluorescently-labeled reaction substrate were added, and compounds I-9 to I-14, II-1, II-2, III-1, and III-2 each were separately added at different concentrations. A blank control group was set. After the reaction was completed, fluorescence analysis was performed. Finally, the results of inhibition of the compounds on the ASM activity were shown in the table below:

Test results of inhibition of compounds I-9 to I-14, II-1, II-2, III-1, and III-2 of the present disclosure on the ASM activity

| Compound | ASM activity inhibition | |
|---|---|---|
| | 10 µM | 1 µM |
| I-9 | 93% | 80% |
| I-10 | 50% | 20% |
| I-11 | 83% | 8% |
| I-12 | 82% | 70% |
| I-13 | 22% | 14% |
| I-14 | 76% | 24% |
| II-1 | 91% | 74% |
| II-2 | 88% | 69% |
| III-1 | 80% | 61% |
| III-2 | 72% | 43% |

### Example 5 Assay of Anti-depression Activity of the Active Compound I-1

In the present disclosure, a chronic depression rat model was established by reserpine (reference: NeurotoxRes, 2014, doi: 10.1007/s12640-013-9454-8). Specific experimental steps were as follows:

In a normal group, 8 rats were randomly selected and raised normally. Rats in the remaining groups each were injected intraperitoneally with reserpine at 0.2 mg/kg once every day for three days. Rats in the remaining groups each were intraperitoneally injected with the compound I-1 at different doses (3 mg/kg, 6 mg/kg, and 12 mg/kg), a positive drug amitriptyline (6 mg/kg), or no drug. Two weeks after the administration, the OFT was performed (reference: China Pharmacy, 2016, 27 (19): 2697-2699), and after the OFT was completed, the SPT was performed.

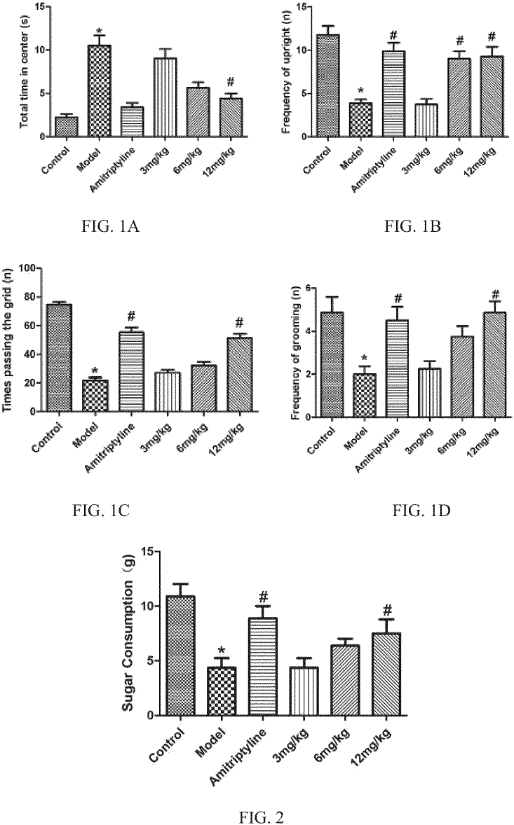

As shown in FIGS. 1A-1D, OFT results showed that, after the rats were administered with the compound I-1, a total time in center, a times passing the grid, a frequency of upright, and a frequency of grooming of the rats were significantly improved. Behavioral indexes of rats administered with high-dose (12 mg/kg) I-1 in the OFT were comparable to that of rats in the amitriptyline control group (6 mg/kg). The above data showed that the compound I-1 exhibited a significant anti-depression activity.

After the OFT was completed, all rats were first trained to drink 10 g/L sucrose-containing water. That is, tap water was replaced with 10 g/L sucrose-containing water within the first 48 h, then the water and food were deprived for 20 h, and then 10 g/L sucrose-containing water was provided for drinking and the drinking amount within 24 h was calculated.

As shown in FIG. 2, SPT results showed that, after the rats were administered with I-1, an uptake capacity of the rat for sucrose-containing water could be significantly restored, where an effect of the high-dose I-1 group (12 mg/kg) was slightly lower than that of the positive control drug amitriptyline (6 mg/kg), but was significantly different from that of the model group (p < 0.01), indicating that the compound I-1 exhibited a specified anti-depression activity in the SPT.

### Example 6 Anti-AS Effect of Compound I-1

ApoE is an important component of plasma lipoproteins, plays an important role in the regulation of a plasma cholesterol level, and is an important molecular target for the occurrence and development of hyperlipidemia, AS, or the like. The development of AS lesions in ApoE-knockout mice is very similar to that in humans. At present, ApoE-knockout mice have become one of the most important animal models for studying the pathogenesis of AS and the anti-AS pharmacology.

ApoE-knockout mice were raised with a high-fat diet for 8 weeks to establish an AS model (mice raised with a high-fat diet are commonly used as an animal model for studying AS, and a modeling method can be seen in (Chinese Journal of Integrative Medicine, 2019, 25 (02): 108-115.).

(1) After mice were raised with a Western diet (21% fat + 0.15% cholesterol) for 12 weeks, a blood lipid level in the mice was increased significantly, and at an aortic root, a vessel wall was thickened, and plaques were also significantly increased, indicating typical pathological features of AS.

(2) After ApoE⁻/⁻ mice were raised with a high-fat diet (including: 18% hydrogenated cocoa butter, 0.15% cholesterol, 7% casein, 7% sucrose, and 3% maltodextrin) for 8 weeks, an aortic valve of the heart of the mice was stained with hematoxylin-eosin (HE) to confirm the successful preparation of the AS model (A detection method can be seen in Biomedicine & Pharmacotherapy, 2018, 97.).

(3) After mice were raised with a high-fat diet for 2 weeks, it was found 8 weeks after right carotid artery cannulation that TC, triacylglycerol (TG), and low-density lipoprotein cholesterol (LDL-C) were significantly increased, and obvious AS plaques were formed at a common carotid artery cannulation site (A dissection method can be seen in Journal of Anatomy, 2018, 41 (01): 16-19). The mice were randomly divided into 6 groups, with 5 mice in each group. The groups were intraperitoneally injected respectively with a blank solvent, a blank solvent, 12 mg/kg compound I-1, and 40 mg/kg compound I-1 once every day for 8 weeks. After the experiment was completed, the animals were sacrificed and main arteries were collected.

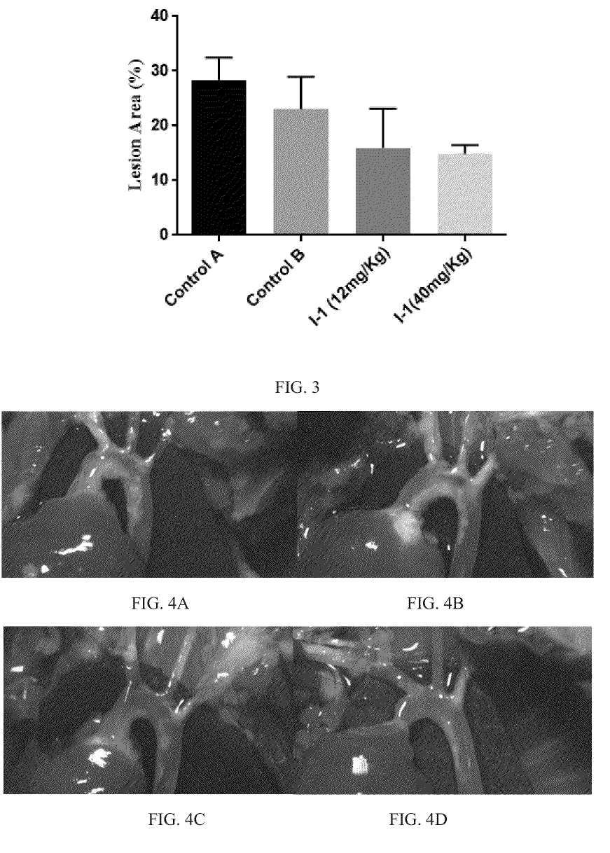

The main arterial vessels were directly observed and photographed under a microscope. Results were shown in FIG. 3 and FIGS. 4A-4D, and it can be seen that obvious plaques were formed at an aortic arch in the non-administration group, and a plaque area was significantly reduced after the administration of the compound I-1 at different doses.

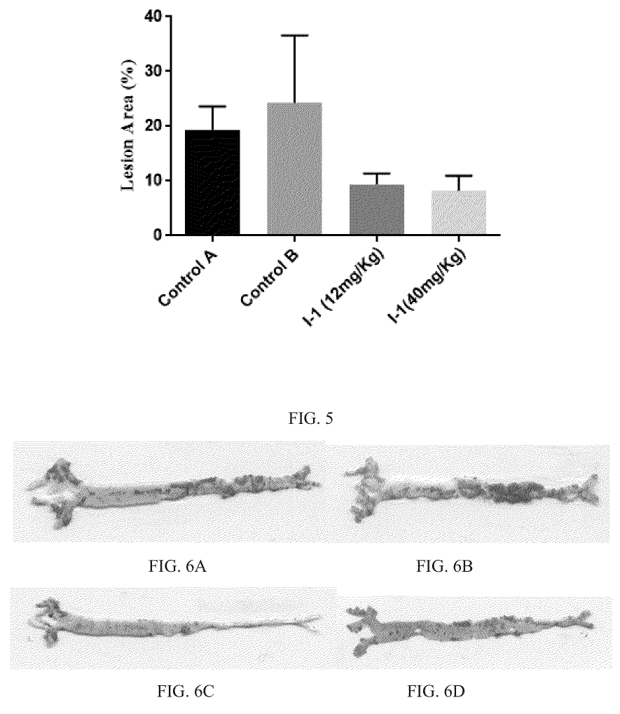

The collected arterial vessels were subjected to lipid staining with ORO and then photographed under a microscope, and image analysis was performed. Results were shown in FIG. 5 and FIGS. 6A-6D, it can be seen that the compound I-1 could significantly reduce a plaque area of AS at 12 mg/kg and 40 mg/kg without dose dependence as a whole.

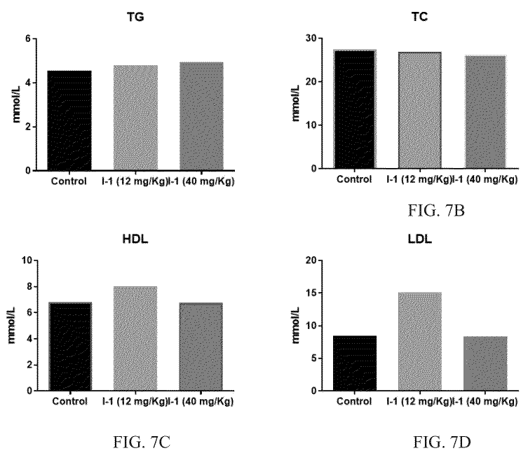

The plasma lipid indexes were analyzed, and results were shown in FIGS. 7A-7D. Compared with the non-administration Control group, the compound I-1 had no impact on the total cholesterol, total TG, and HDL in plasma, but significantly increased an LDL level.

What is claimed is:

1. A hydroxamic acid-containing compound with a structure represented by formula I: 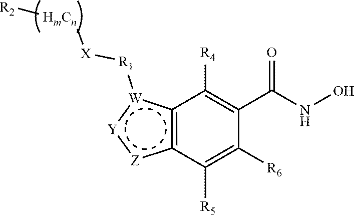
or a pharmaceutically acceptable salt or a prodrug of the compound represented by the formula I, wherein
R₁ is selected from the group consisting of C₁-C₁₀ saturated linear alkyl, C₁-C₁₀ unsaturated linear alkyl, C₃-C₁₀ saturated branched alkyl, C₃-C₁₀ unsaturated branched alkyl, C₃-C₁₀ cycloalkyl, and phenyl;
X is selected from the group consisting of C, N, O, and S;
CₙHₘ is selected from the group consisting of C₁-C₄ saturated linear alkyl, C₁-C₄ unsaturated linear alkyl, C₁-C₄ saturated branched alkyl, and C₁-C₄ unsaturated branched alkyl, wherein 1 ≤ n ≤ 4 and 2 ≤ m ≤ 8;
R₂ is a monosubstituted or disubstituted phenyl group, a substituent in the R₂ is located at an ortho, meta, or para position of a benzene ring and is selected from the group consisting of hydrogen, fluoro, chloro, bromo, cyano, OR₃, CF₃, and SF₅; or R₂ is selected from the group consisting of pyridin-2-yl, pyridin-3-yl, pyridin-4-yl, C₁-C₁₀ saturated linear alkyl, C₁-C₁₀ unsaturated linear alkyl, C₃-C₁₀ saturated branched alkyl, and C₃-C₁₀ unsaturated branched alkyl, wherein R₃ in OR₃ is selected from the group consisting of H, C₁-C₄ linear or branched alkyl, C₃-C₆ cycloalkyl, and phenyl;
W, Y, and Z are each independently any one selected from the group consisting of C, N, O, and S; and
R₄, R₅, and R₆ are each independently any one selected from the group consisting of H, F, Cl, Br, CF₃, CHF₂, COCF₃, COCH₃, COC₂H₅, CN, C₁-C₆ alkoxy, C₁-C₆ saturated linear alkyl, C₁-C₆ unsaturated linear alkyl, C₃-C₆ saturated branched alkyl, C₃-C₆ unsaturated branched alkyl, and C₃-C₆ cycloalkyl.

2. The hydroxamic acid-containing compound according to claim 1, wherein R₁ is selected from the group consisting of phenyl, propyl, and n-butyl; R₂ is selected from the group consisting of 4-chlorophenyl, 4-trifluoromethylphenyl, 3-trifluoromethylphenyl, 4-chloro-3-trifluoromethyl-phenyl, and 2,4-bis(trifluoromethyl)-phenyl; X is C or O; W or Z is N; Y is C; and R₄, R₅, and R₆ each are H.

3. The hydroxamic acid-containing compound according to claim 1, wherein the hydroxamic acid-containing compound has a structure selected from the group consisting of 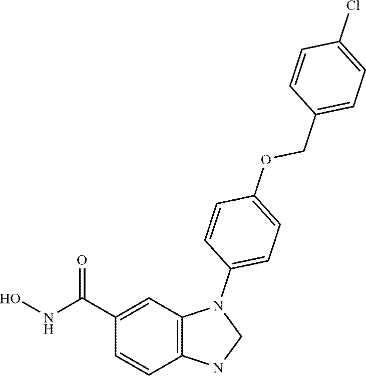 [SMILES: O=C(NO)c1ccc2c(c1)N(c1ccc(OCc3ccc(Cl)cc3)cc1)CN2] 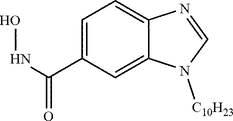 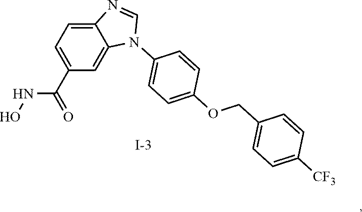 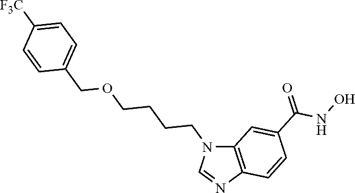 [SMILES: O=C(NO)c1ccc2ncn(CCCCOCc3ccc(C(F)(F)F)cc3)c2c1] 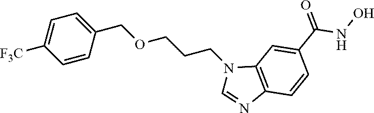 [SMILES: O=C(NO)c1ccc2ncn(CCCOCc3ccc(C(F)(F)F)cc3)c2c1] 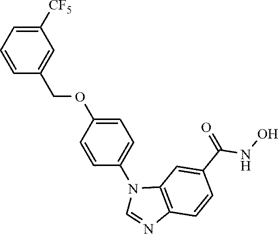 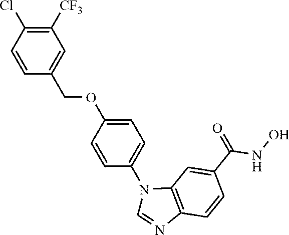 [SMILES: O=C(NO)c1ccc2ncn(-c3ccc(OCc4ccc(Cl)c(C(F)(F)F)c4)cc3)c2c1] 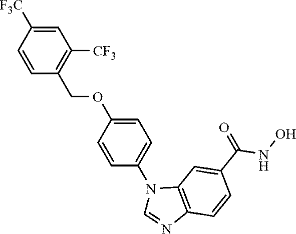 [SMILES: O=C(NO)c1ccc2ncn(-c3ccc(OCc4ccc(C(F)(F)F)cc4C(F)(F)F)cc3)c2c1] 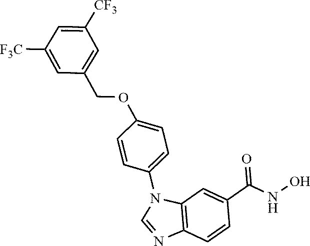 [SMILES: O=C(NO)c1ccc2ncn(-c3ccc(OCc4cc(C(F)(F)F)cc(C(F)(F)F)c4)cc3)c2c1] 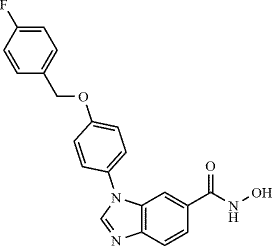 [SMILES: O=C(NO)c1ccc2ncn(-c3ccc(OCc4ccc(F)cc4)cc3)c2c1] 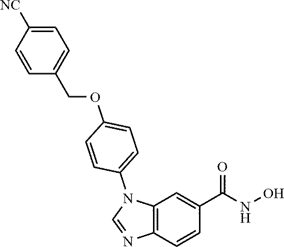 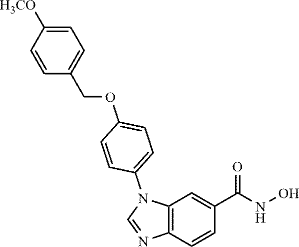 [SMILES: COc1ccc(COc2ccc(-n3cnc4ccc(C(=O)NO)cc43)cc2)cc1] 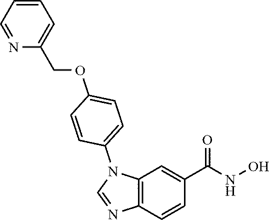 [SMILES: O=C(NO)c1ccc2ncn(-c3ccc(OCc4ccccn4)cc3)c2c1] 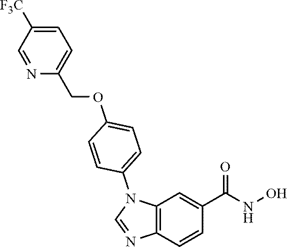 [SMILES: O=C(NO)c1ccc2ncn(-c3ccc(OCc4ccc(C(F)(F)F)cn4)cc3)c2c1] 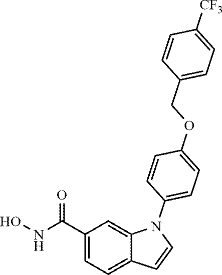 [SMILES: O=C(NO)c1ccc2ccn(-c3ccc(OCc4ccc(C(F)(F)F)cc4)cc3)c2c1] 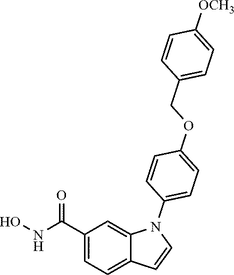 [SMILES: COc1ccc(COc2ccc(-n3ccc4ccc(C(=O)NO)cc43)cc2)cc1] 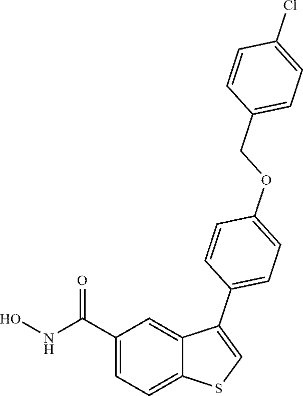 [SMILES: O=C(NO)c1ccc2scc(-c3ccc(OCc4ccc(Cl)cc4)cc3)c2c1]
and 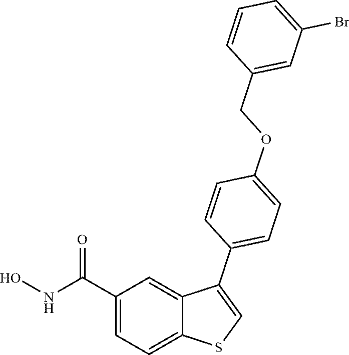 [SMILES: O=C(NO)c1ccc2scc(-c3ccc(OCc4cccc(Br)c4)cc3)c2c1]
.

4. A method of a use of the hydroxamic acid-containing compound according to claim 1 in a preparation of an acid sphingomyelinase (ASM) inhibitor.

5. A method a use of the hydroxamic acid-containing compound according to claim 1 in a preparation of a drug for treating a disease selected from the group consisting of atherosclerosis (AS), diabetes, pulmonary emphysema, pulmonary edema, pulmonary fibrosis, chronic obstructive pulmonary disease (COPD), pulmonary hypertension, cystic fibrosis, non-alcoholic fatty liver disease (NAFLD), Alzheimer’s disease (AD), multiple sclerosis (MS), stroke, and depression.

6. The hydroxamic acid-containing compound according to claim 2, wherein the hydroxamic acid-containing compound has a structure selected from the group consisting of 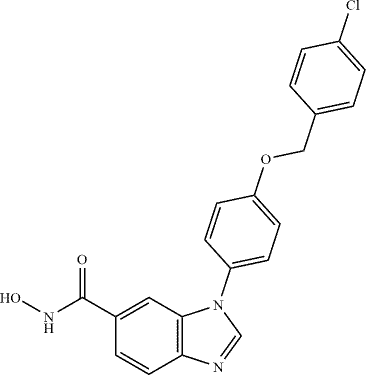 [SMILES: O=C(NO)c1ccc2ncn(-c3ccc(OCc4ccc(Cl)cc4)cc3)c2c1] 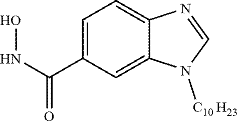 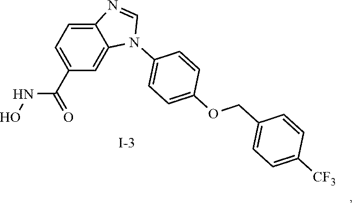 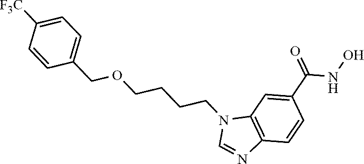 [SMILES: O=C(NO)c1ccc2ncn(CCCCOCc3ccc(C(F)(F)F)cc3)c2c1] 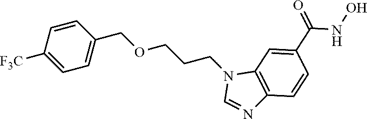 [SMILES: O=C(NO)c1ccc2ncn(CCCOCc3ccc(C(F)(F)F)cc3)c2c1] 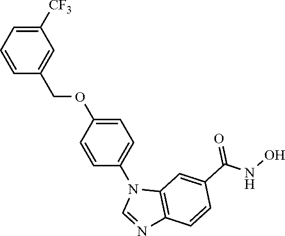 [SMILES: O=C(NO)c1ccc2ncn(-c3ccc(OCc4cccc(C(F)(F)F)c4)cc3)c2c1] 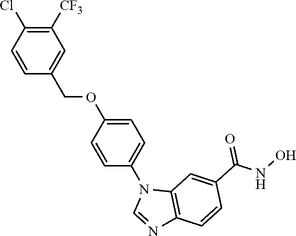 [SMILES: O=C(NO)c1ccc2ncn(-c3ccc(OCc4ccc(Cl)c(C(F)(F)F)c4)cc3)c2c1] 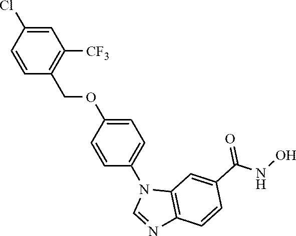 [SMILES: O=C(NO)c1ccc2ncn(-c3ccc(OCc4ccc(Cl)cc4C(F)(F)F)cc3)c2c1] 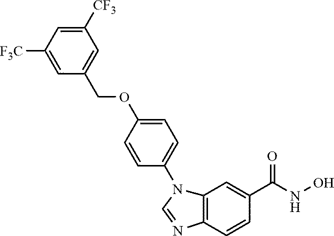 [SMILES: O=C(NO)c1ccc2ncn(-c3ccc(OCc4cc(C(F)(F)F)cc(C(F)(F)F)c4)cc3)c2c1] 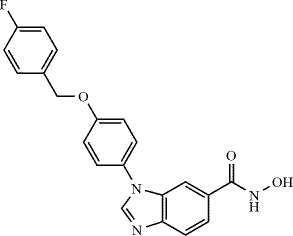 [SMILES: O=C(NO)c1ccc2ncn(-c3ccc(OCc4ccc(F)cc4)cc3)c2c1] 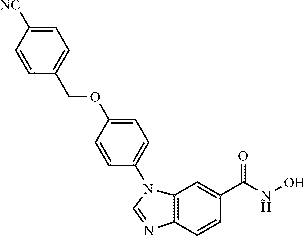 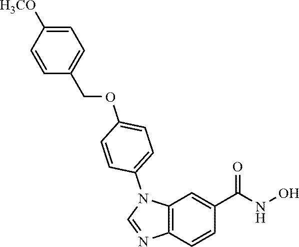 [SMILES: COc1ccc(COc2ccc(-n3cnc4ccc(C(=O)NO)cc43)cc2)cc1] 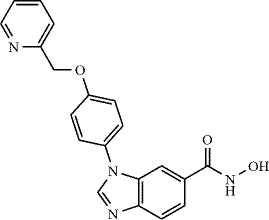 [SMILES: O=C(NO)c1ccc2ncn(-c3ccc(OCc4ccccn4)cc3)c2c1] 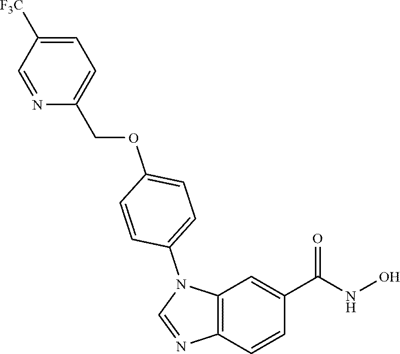 [SMILES: O=C(NO)c1ccc2ncn(-c3ccc(OCc4ccc(C(F)(F)F)cn4)cc3)c2c1] 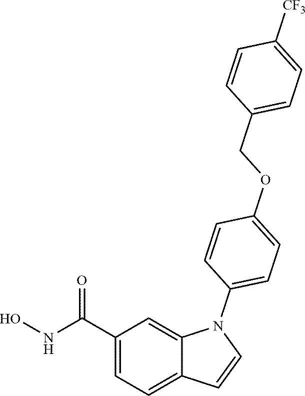 [SMILES: O=C(NO)c1ccc2ccn(-c3ccc(OCc4ccc(C(F)(F)F)cc4)cc3)c2c1] 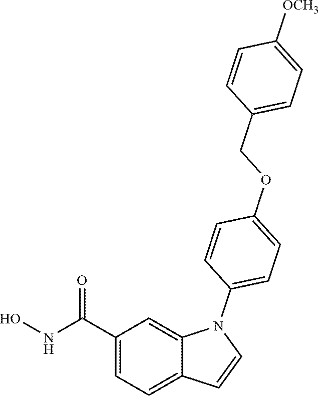 [SMILES: COc1ccc(COc2ccc(-n3ccc4ccc(C(=O)NO)cc43)cc2)cc1] 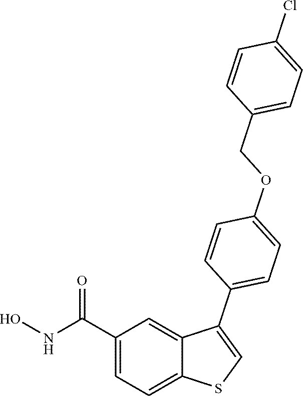 [SMILES: O=C(NO)c1ccc2scc(-c3ccc(OCc4ccc(Cl)cc4)cc3)c2c1]
and 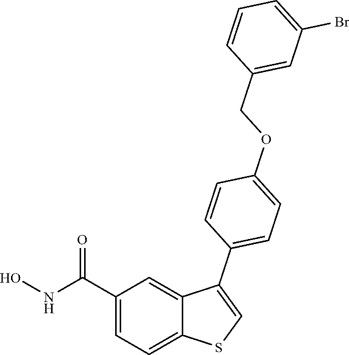 [SMILES: O=C(NO)c1ccc2scc(-c3ccc(OCc4cccc(Br)c4)cc3)c2c1]
.

7. The method according to claim 4, wherein R₁ is selected from the group consisting of phenyl, propyl, and n-butyl; R₂ is selected from the group consisting of 4-chlorophenyl, 4-trifluoromethylphenyl, 3-trifluoromethylphenyl, 4-chloro-3-trifluoromethyl-phenyl, and 2,4-bis(trifluoromethyl)-phenyl; X is C or O; W or Z is N; Y is C; and R₄, R₅, and R₆ each are H.

8. The method according to claim 4, wherein the hydroxamic acid-containing compound has a structure selected from the group consisting of 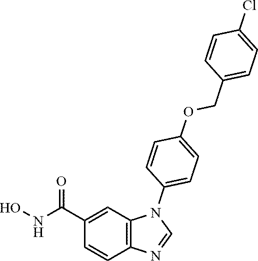 [SMILES: O=C(NO)c1ccc2ncn(-c3ccc(OCc4ccc(Cl)cc4)cc3)c2c1] 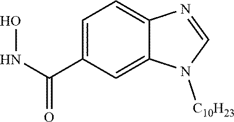 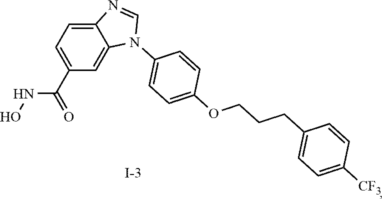 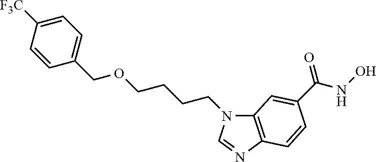 [SMILES: O=C(NO)c1ccc2ncn(CCCCOCc3ccc(C(F)(F)F)cc3)c2c1] 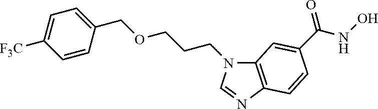 [SMILES: O=C(NO)c1ccc2ncn(CCCOCc3ccc(C(F)(F)F)cc3)c2c1] 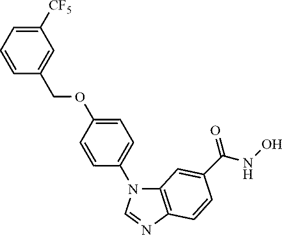 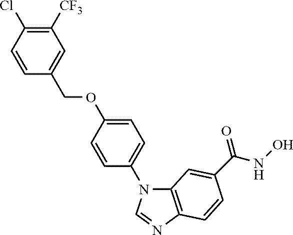 [SMILES: O=C(NO)c1ccc2ncn(-c3ccc(OCc4ccc(Cl)c(C(F)(F)F)c4)cc3)c2c1] 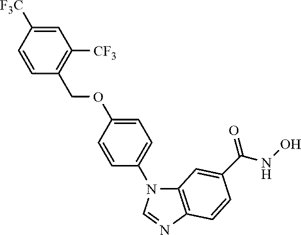 [SMILES: O=C(NO)c1ccc2ncn(-c3ccc(OCc4ccc(C(F)(F)F)cc4C(F)(F)F)cc3)c2c1] 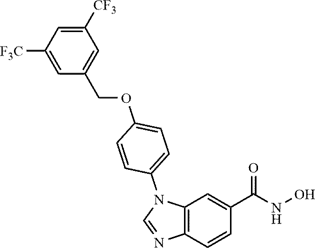 [SMILES: O=C(NO)c1ccc2ncn(-c3ccc(OCc4cc(C(F)(F)F)cc(C(F)(F)F)c4)cc3)c2c1] 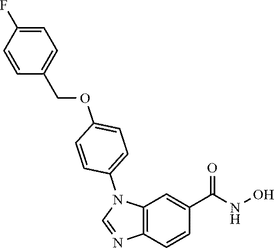 [SMILES: O=C(NO)c1ccc2ncn(-c3ccc(OCc4ccc(F)cc4)cc3)c2c1] 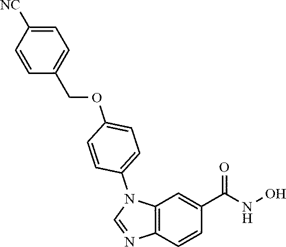 [SMILES: N#Cc1ccc(COc2ccc(-n3cnc4ccc(C(=O)NO)cc43)cc2)cc1] 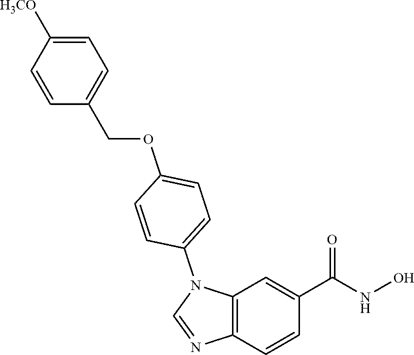 [SMILES: COc1ccc(COc2ccc(-n3cnc4ccc(C(=O)NO)cc43)cc2)cc1] 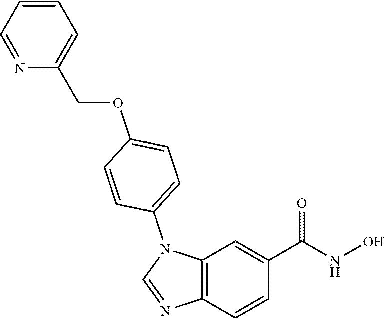 [SMILES: O=C(NO)c1ccc2ncn(-c3ccc(OCc4ccccn4)cc3)c2c1] 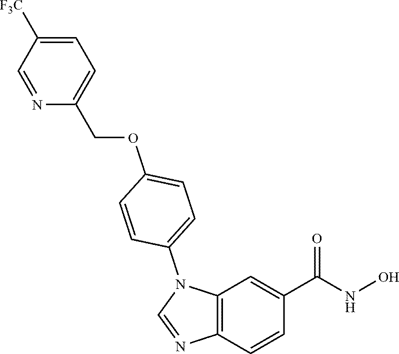 [SMILES: O=C(NO)c1ccc2ncn(-c3ccc(OCc4ccc(C(F)(F)F)cn4)cc3)c2c1] 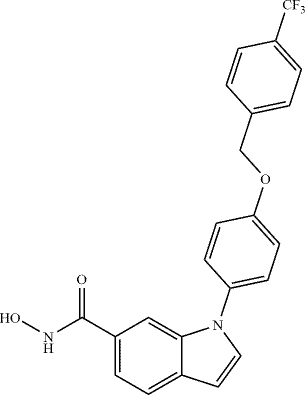 [SMILES: O=C(NO)c1ccc2ccn(-c3ccc(OCc4ccc(C(F)(F)F)cc4)cc3)c2c1] 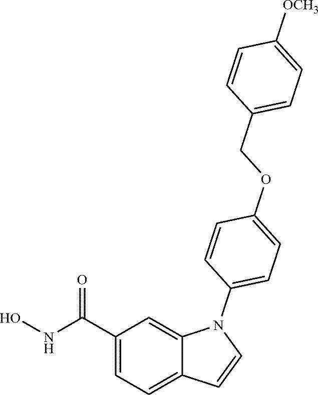 [SMILES: COc1ccc(COc2ccc(-n3ccc4ccc(C(=O)NO)cc43)cc2)cc1] 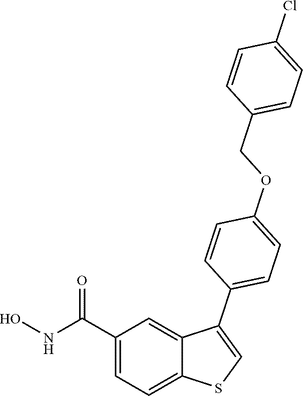 [SMILES: O=C(NO)c1ccc2scc(-c3ccc(OCc4ccc(Cl)cc4)cc3)c2c1]
and 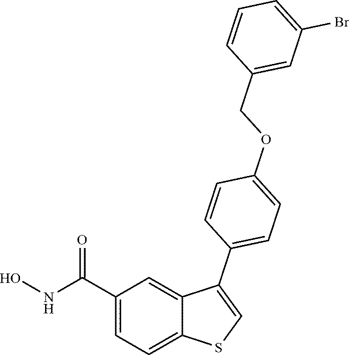 [SMILES: O=C(NO)c1ccc2scc(-c3ccc(OCc4cccc(Br)c4)cc3)c2c1]
.

9. The method according to claim 5, wherein R₁ is selected from the group consisting of phenyl, propyl, and n-butyl; R₂ is selected from the group consisting of 4-chlorophenyl, 4-trifluoromethylphenyl, 3-trifluoromethylphenyl, 4-chloro-3-trifluoromethyl-phenyl, and 2,4-bis(trifluoromethyl)-phenyl; X is C or O; W or Z is N; Y is C; and R₄, R₅, and R₆; each are H.

10. The method according to claim 5, wherein the hydroxamic acid-containing compound has a structure selected from the group consisting of 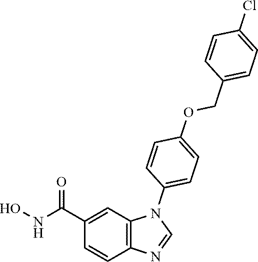 [SMILES: O=C(NO)c1ccc2ncn(-c3ccc(OCc4ccc(Cl)cc4)cc3)c2c1] 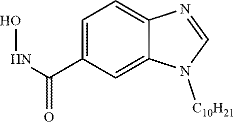 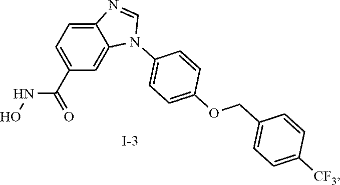 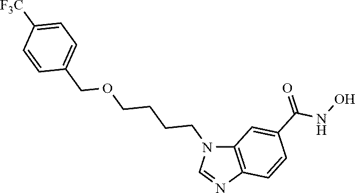 [SMILES: O=C(NO)c1ccc2ncn(CCCCOCc3ccc(C(F)(F)F)cc3)c2c1] 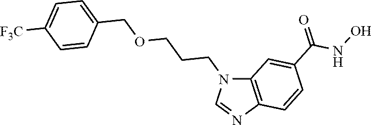 [SMILES: O=C(NO)c1ccc2ncn(CCCOCc3ccc(C(F)(F)F)cc3)c2c1] 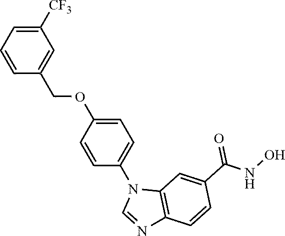 [SMILES: O=C(NO)c1ccc2ncn(-c3ccc(OCc4cccc(C(F)(F)F)c4)cc3)c2c1] 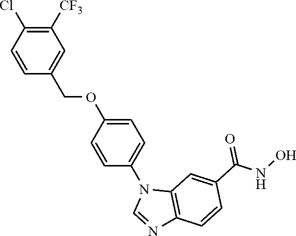 [SMILES: O=C(NO)c1ccc2ncn(-c3ccc(OCc4ccc(Cl)c(C(F)(F)F)c4)cc3)c2c1] 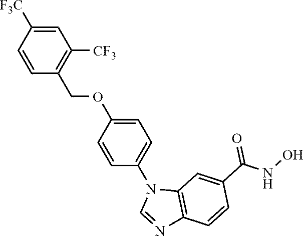 [SMILES: O=C(NO)c1ccc2ncn(-c3ccc(OCc4ccc(C(F)(F)F)cc4C(F)(F)F)cc3)c2c1] 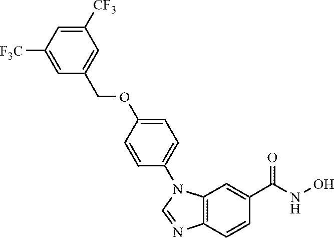 [SMILES: O=C(NO)c1ccc2ncn(-c3ccc(OCc4cc(C(F)(F)F)cc(C(F)(F)F)c4)cc3)c2c1] 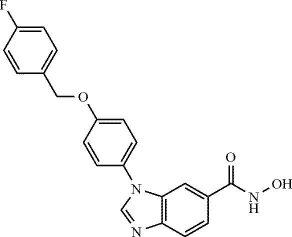 [SMILES: O=C(NO)c1ccc2ncn(-c3ccc(OCc4ccc(F)cc4)cc3)c2c1] 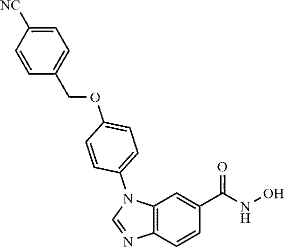 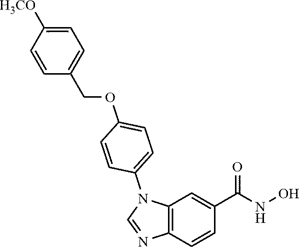 [SMILES: COc1ccc(COc2ccc(-n3cnc4ccc(C(=O)NO)cc43)cc2)cc1] 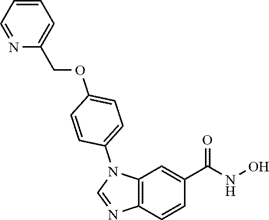 [SMILES: O=C(NO)c1ccc2ncn(-c3ccc(OCc4ccccn4)cc3)c2c1] 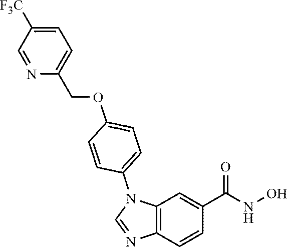 [SMILES: O=C(NO)c1ccc2ncn(-c3ccc(OCc4ccc(C(F)(F)F)cn4)cc3)c2c1] 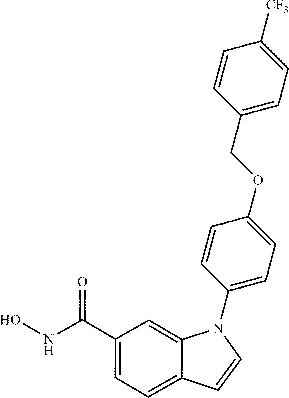 [SMILES: O=C(NO)c1ccc2ccn(-c3ccc(OCc4ccc(C(F)(F)F)cc4)cc3)c2c1] 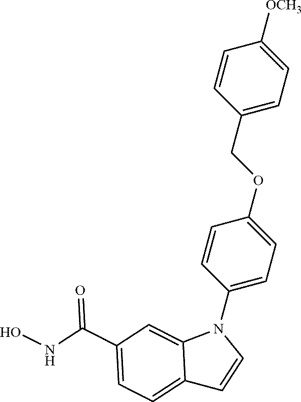 [SMILES: COc1ccc(COc2ccc(-n3ccc4ccc(C(=O)NO)cc43)cc2)cc1] 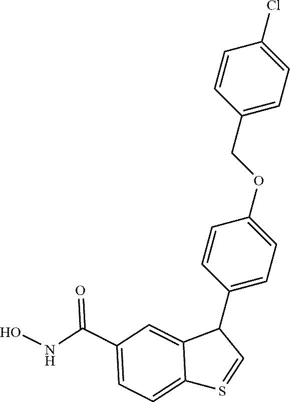
and 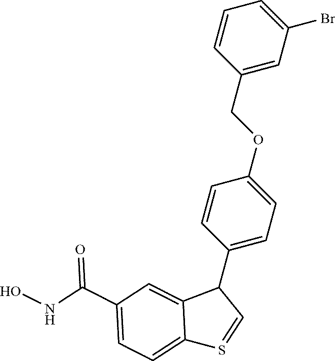
.
